(19) United States
(12) Patent Application Publication
(10) Pub. No.: US 2019/0191846 A1
(43) Pub. Date: Jun. 27, 2019

(54) COMPOSITIONS FOR MANICURES AND NAIL ENHANCEMENTS AND METHODS OF USE

(71) Applicant: Quy That Ton, Baton Rouge, LA (US)

(72) Inventor: Quy That Ton, Baton Rouge, LA (US)

(21) Appl. No.: 16/182,587

(22) Filed: Nov. 6, 2018

Related U.S. Application Data
(60) Provisional application No. 62/582,194, filed on Nov. 6, 2017.

(51) Int. Cl. A45D 31/00; A61K 8/81; A61Q 3/02

(52) U.S. Cl. CPC A45D 31/00; A61K 2800/43; A61Q 3/02; A61K 8/8152

(57) ABSTRACT

Example embodiments of the compositions for manicures and nail enhancements and methods of use disclosed herein include applying a colored base coat and dipping the nails in a clear acrylic powder. Instead of creating hundreds of colors of powder, the dipping glue/powder manicure and nail enhancement may be accomplished by applying colored base coats on a nail of a finger (or toe) and then dipping the nail into clear acrylic powder or applying clear acrylic powder onto base coat. Colored base coats 120 may be made of a glue base including a pigment and other ornamental attributes. The glue base may include cyanoacrylate.

### CROSS-REFERENCE TO RELATED APPLICATION

This application claims benefit to U.S. provisional patent application Ser. No. 62/582,194, filed on Nov. 6, 2017, which is incorporated by reference herein.

### TECHNICAL FIELD

The present disclosure is generally related to manicures and, more particularly, is related to compositions for manicures and nail enhancements and methods of their use.

### BACKGROUND

Some types of manicures are French manicures, hot oil manicures, and dip powder manicures. French manicures typically use artificial nails that are painted and polished to resemble natural nails, or can be accomplished on natural nails. The hot oil manicure typically cleans cuticles and softens them with oils that can be mineral oil, olive oil, some lotions, or commercial preparations in an electric heater.

The artificial French manicures can be accomplished with acrylics and gel polish where the nails are painted with coats of liquid polish. There are heretofore unaddressed needs with previous solutions.

### SUMMARY

Example embodiments of the present disclosure provide a composition for manicures and nail enhancements. Briefly described, one example embodiment of the composition, among others, can be implemented as follows: a first pigmented glue base coat configured to be applied to a nail; and a clear acrylic powder coat configured to be applied to the first pigmented glue base coat.

Embodiments of the present disclosure can also be viewed as providing methods for using compositions for manicures and nail enhancements. In this regard, one embodiment of such a method, among others, can be broadly summarized by the following steps: applying a first pigmented glue base coat to a nail; and applying a clear acrylic powder to the pigmented glue base coat.

### BRIEF DESCRIPTION OF THE DRAWINGS

FIG. 1 is a diagram of an example embodiment of a composition for manicures and nail enhancements.

FIG. 2 is a diagram of an example embodiment of a multiple area application of the composition of FIG. 1.

FIG. 3 is a flow diagram of an example embodiment of a method of using the compositions for manicures and nail enhancements of FIG. 1.

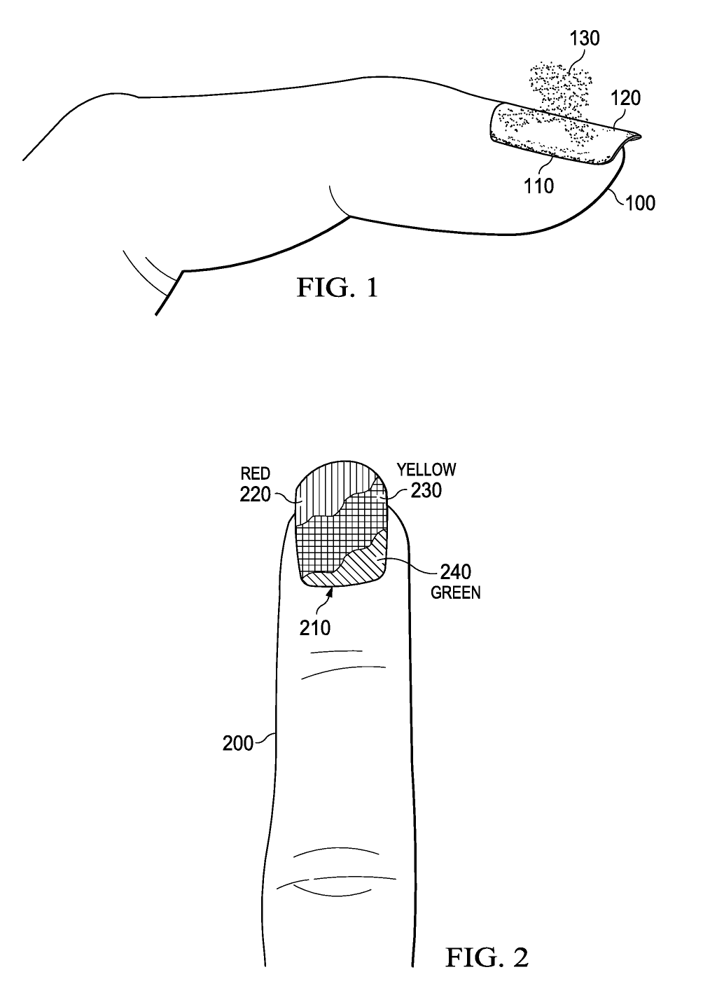

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

Example embodiments of the compositions for manicures and nail enhancements and methods of use disclosed herein include applying a colored glue base coat and dipping the nails in a clear acrylic powder. Instead of creating hundreds of colors of powder, the dipping glue/powder manicure and nail enhancement may be accomplished by applying colored base coats 120 on nail 110 of finger (or toe) 100 and then dipping nail 110 into clear acrylic powder 130 or applying clear acrylic powder 130 onto base coat 120. Colored base coats 120 may be made of a glue base including a pigment and other ornamental attributes. The glue base may include cyanoacrylate.

In an example embodiment of the disclosed dipping glue/powder manicure and nail enhancement, nail technicians stock different colors of the glue (base) solution 120 and one (1) clear acrylic powder 130 to dip or apply. Nail technicians may perform traditional dipping glue/powder manicure and nail enhancement procedures with colored base 120 and powder dipped coat 130.

In an example embodiment, colored or pigmented glue base coat 120 is wet in order for clear powder 130 to be absorbed. A second glue base coat 120 with the same pigment (or different pigment or ornamentality) may be applied to nail 110 and then re-dipped into clear acrylic powder 130. This builds thickness and color uniformity. Additional coats may be applied.

In an example embodiment, the dipping glue/powder manicure and nail enhancement is applied to a real nail of a person. In an alternative embodiment, the dipping glue/powder manicure and nail enhancement is applied to a fake nail and then the enhanced nail is applied onto the person's real nail.

An example ornamental pattern is provided in FIG. 2 with finger 200, nail 210, first nail area 220, second nail area 230, and third nail area 240. In this example, first nail area 220 is painted red, second nail area 230 is painted yellow, and third nail area 240 is painted green. The three nail areas are used for example illustrative purposes only. Any number of and shape of the areas may be used.

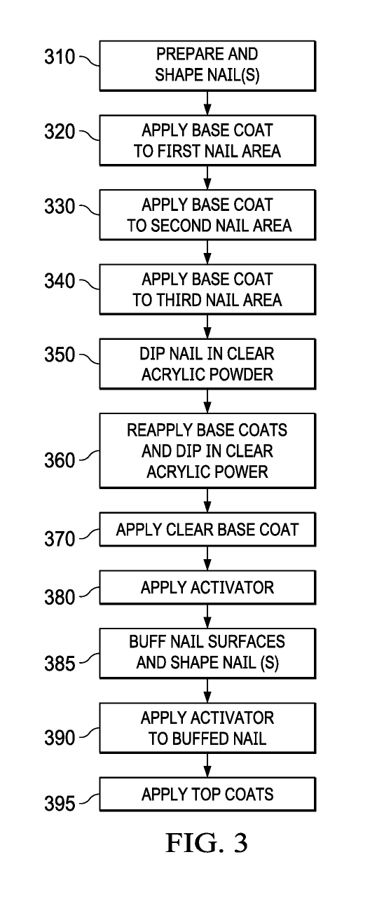

In an example embodiment of a method of using a pigmented glue base coat and clear acrylic powder manicure composition as provided in FIG. 3, in step 310, the nails are prepared and shaped. In step 320, red pigmented glue base coat is applied to first nail area 220. In step 330, yellow pigmented glue base coat is applied to second nail area 230. In step 340, green pigmented glue base coat is applied to third nail area 240. The order of the application of the base coats may be modified. In step 350, nail 210 is dipped in clear acrylic powder. In an example embodiment, the pigmented glue base coat is wet to allow the absorption by or adherence of the clear acrylic powder to the glue base coat.

In an example embodiment, each of the three nail areas 220, 230, 240 are coated in a pigmented glue base coat and the clear acrylic powder is absorbed in each of the three nail areas. In an alternative embodiment at least one of the multiple nail areas does not have glue in the base coat and doesn't absorb the clear acrylic powder, whereas at least one of the other multiple nail areas does include glue in the base coat and absorbs the clear acrylic powder.

In step 360, the base coats are reapplied and the nails are again dipped in the clear acrylic powder to build thickness and color uniformity. In an example embodiment, in step 370, a clear glue base coat is applied. In step 380, an activator is applied to dry the manicure and nail enhancement composition. In step 385, the nail surfaces are buffed and the nails are shaped. In step 390, activator is applied to the buffed nails. In step 395, one or more coats of top coat are applied.

The flow chart of FIG. 3 shows the architecture, functionality, and operation of a possible implementation of the dipping glue/powder manicure and nail enhancement process. In this regard, each block represents a step of the process. It should also be noted that in some alternative implementations, the functions noted in the blocks may occur out of the order noted in FIG. 3. For example, two blocks shown in succession in FIG. 3 may in fact be executed substantially concurrently or the blocks may sometimes be executed in the reverse order, depending upon the functionality involved.

Although the present disclosure has been described in detail, it should be understood that various changes, substitutions and alterations can be made thereto without departing from the spirit and scope of the disclosure as defined by the appended claims.

Therefore, at least the following is claimed:

1. A method comprising:
applying a first pigmented glue base coat to a nail; and
applying a clear acrylic powder to the pigmented glue base coat.

2. The method of claim 1, wherein the applying of the clear acrylic powder comprises at least one of dipping and dusting.

3. The method of claim 1, further comprising:
applying a second glue base coat; and
dipping in the clear acrylic powder.

4. The method of claim 3, wherein the second glue base coat is the same pigment and ornamentality as the first glue base coat.

5. The method of claim 3, wherein the second glue base coat is different in pigment and/or ornamentality from the first glue base coat.

6. The method of claim 1, wherein the nail is a real human nail.

7. The method of claim 1, wherein the nail is a fake nail and further comprising applying the enhanced fake nail to a real nail.

8. A device, comprising:
a fake nail configured to be applied to a real nail;
a first pigmented glue base coat configured to be applied to the fake nail; and
a clear acrylic powder configured to be applied to the pigmented glue base coat.

9. The device of claim 8, wherein the clear acrylic powder is applied to the pigmented glue base coat by at least one of dipping and dusting.

10. The device of claim 8, further comprising a second glue base coat configured to be applied to the fake nail.

11. The device of claim 10, wherein the second glue base coat is the same pigment and ornamentality as the first glue base coat.

12. The device of claim 10, wherein the second glue base coat is different in pigment and/or ornamentality from the first glue base coat.

13. A composition, comprising:
a first pigmented glue base coat configured to be applied to a nail; and
a clear acrylic powder coat configured to be applied to the first pigmented glue base coat.

14. The composition of claim 13, wherein the clear acrylic powder is applied to the first pigmented glue base coat by at least one of dipping and dusting.

15. The composition of claim 13, further comprising a second glue base coat configured to be applied to the nail.

16. The composition of claim 15, wherein the second glue base coat is the same pigment and ornamentality as the first pigmented glue base coat.

17. The composition of claim 15, wherein the second glue base coat is different in pigment and/or ornamentality from the first pigmented glue base coat.

18. The composition of claim 15, further comprising a clear base coat.

19. The composition of claim 18, further comprising a first activator coat.

20. The composition of claim 19, further comprising:
a second activator coat configured to be applied after the nail has been buffed; and
at least one top coat.
